(19) United States
(12) Patent Application Publication
(10) Pub. No.: US 2020/0100890 A1
(43) Pub. Date: Apr. 2, 2020

(54) BONE ANCHOR

(71) Applicant: Spectrum Health Innovations, LLC, Grand Rapids, MI (US)

(72) Inventors: Kendall D. Hamilton, Ada, MI (US); Alissa M. Smith, Coldwater, MI (US)

(21) Appl. No.: 16/572,174

(22) Filed: Sep. 16, 2019

Related U.S. Application Data
(60) Provisional application No. 62/739,468, filed on Oct. 1, 2018.

(51) Int. Cl. A61F 2/08

(52) U.S. Cl. CPC A61F 2/0811; A61F 2/30756; A61F 2002/0894

(57) ABSTRACT

A bone anchor including an outer sleeve defining slots and an inner sleeve including fins extendable radially outward through the slots to engage surrounding cancellous bone. The outer sleeve includes an outward extending flange disposed about an opening defined by the outer sleeve configured to receive a fastener. The flange can seat against cortical bone around an opening sized for the anchor. The anchor can be a suture anchor including the fastener that engages the inner sleeve when installed therein and pushes the fins outward to engage the cancellous bone as the fastener is installed, better securing the anchor to the bone, particularly where the bone is osteoporotic, weak, broken, diseased, and/or compromised. The outer sleeve can include projections between fins to prevent rotation as the fastener is installed. A related method of using the suture anchor is provided to anchor soft tissue with a suture to bone.

### BACKGROUND OF THE INVENTION

The present invention relates to bone anchors, and more particularly to a bone anchor constructed to sufficiently engage bone that may be weak, soft, osteoporotic or otherwise compromised.

Bone anchors are utilized in numerous surgical procedures to secure certain elements to bone. Some bone anchors are used in arthroscopic and endoscopic surgical procedures to permanently attach a suture to bone tissue. For example, in procedures that require the suturing of soft tissue, such as tendons, ligaments, cartilage or muscle, to bone tissue, a suture typically anchors the soft tissue to the bone using either knotless or knotted technology. Most such suture anchors are constructed to be secured in predrilled holes or tunnels in the bone.

Typically, suture anchors are the form of threaded screws having a head with a loop through which a suture can be threaded. To install the suture anchor, a pilot hole is drilled into the bone tissue. In some cases, the pilot hole is tapped to receive the threads of the suture anchor screw. In some cases, where a surgeon determines that the pilot hole is not ideally located, another pilot hole may need to be drilled and tapped, which can require additional steps in an already lengthy procedure. In other cases, a surgeon may determine that the bone around the pilot hole is compromised so that another pilot hole must be formed in a better location for anchoring the screw.

Compromised bone can become more problematic when suture anchors are used in certain procedures. For example, during a rotator cuff repair surgery, multiple suture anchors are utilized and joined with underlying bone to secure torn rotator cuff tendons permanently to that bone. In such a rotator cuff repair surgery, suture anchors, in the form of screws, are effectively screwed directly into bone tissue, for example, into pilot holes or punctures made in the humeral head. These suture anchors typically include a loop opposite the tip of the screw, with threads extending between the tip and the loop. The screw is threaded into the bone puncture a preselected distance such that the suture limbs remains accessible. A suture is threaded through the loop and stitched through the end of the torn rotator cuff tendons. The suture is tensioned to secure the tendons in place. In such a procedure, there typically are multiple separate suture anchors that are installed in multiple separate locations in the bone, depending on the size of the tear.

Although such conventional suture anchors work relatively well, they suffer shortcomings when the surrounding bone is compromised. For example, where the bone is osteoporotic, weak, broken or diseased, or compromised due to holes from previous surgeries, the bone in that area is considered soft. When the bone is soft and a puncture is made to accommodate a suture anchor, oftentimes the hole created is larger than the anchor that is installed in the hole. Thus, the anchor may not secure well in that hole. Such complications and inadequate installation of the suture anchors can lead to an inadequate and unstable fixation. Other times, the suture anchor will be anchored in bone tissue too soft to securely hold the anchor, in which case, the suture anchor can be partially or fully pulled out from the hole and bone after the surgery, leading to failure of the repair construct. Frequently, additional anchors are needed to anchor the tendon to the soft bone. Other issues that can result from unsatisfactory suture anchoring can include a prolonged recovery time, pain in the area of the anchors and in the other cases, the need for a repeat surgery to more adequately connect the repaired tissue to bone.

Accordingly, there remains room for improvement to enhance the attachment of suture anchors and other bone anchors to bone, particularly bone that has been compromised.

### SUMMARY OF THE INVENTION

A bone anchor is provided including a sleeve defining slots and fins extendable radially outward through the slots to engage surrounding cancellous bone.

In one embodiment, the anchor can be a suture anchor including a fastener that pushes the fins outward to engage cancellous bone as the fastener is installed, better securing the anchor to the bone, particularly where the bone is osteoporotic, weak, broken, diseased or compromised.

In yet another embodiment, the anchor can include an outer sleeve and an inner sleeve inside the outer sleeve. These sleeves can be generally concentric. The outer sleeve can define the slots and the inner sleeve can include the fins.

In another embodiment, the outer sleeve can include an outward extending flange disposed about an opening defined by the outer sleeve configured to receive the fastener. The flange can seat against cortical bone around an opening defined in the bone and sized for the anchor. The flange can be an annular flange and can extend around part or all of the outer sleeve. The flange can have a lower surface that directly engages the outer surface of the cortical bone.

In still another embodiment, the outer sleeve can include stationary projections to prevent rotation of the outer sleeve relative to the bone as a fastener is installed in the anchor, for example as the fastener is rotated, in the interior of the inner sleeve. The projections can be disposed between the fins and/or between the slots in the outer sleeve. The projections can extend a preselected distance from the outer surface of the outer sleeve, which can be less than or equal to the distance away from the outer surface that the fins extend when the fins are in an extended mode.

In yet another embodiment, the inner sleeve can be segmented to include arms. The arms can include and/or can be joined with the fins. The arms can be resiliently and/or flexibly joined with the inner or outer sleeve. The arms can be configured to flex inward when the fins engage bone around an opening for the anchor or as the anchor is inserted into cancellous bone. In this state, or when the arms are static and unflexed, the fins can be in a retracted mode. The arms can be configured to bend outward when the fastener is installed. When the arms bend outward, they can engage and project into surrounding cancellous bone. In this state, the fins can be in an extended mode.

In even another embodiment, the fins can include upper ends that protrude a preselected distance from the outer surface of the outer sleeve in the extended mode. This preselected distance can be such that at least the upper end extends beyond a radius or other dimension of the anchor opening in the bone. Thus, if the anchor is pulled upward to dislodge it from the opening, the upper end of the fin can engage an interior of the cortical bone, thereby impairing such removal.

In yet another embodiment, a fin of the anchor can include an upper end, closer to the outer sleeve opening and a lower end, close to a tip of the outer sleeve. The fin can hinge, flex, bend and/or pivot at one or more locations closer to the tip. The fin can rotate in a vertical plane, within which the longitudinal axis lays, outward and away from the axis, optionally with the upper end increasing in distance away from the longitudinal axis while the lower end remains generally unmoved relative to the longitudinal axis.

In a further embodiment, a method is provided including: inserting into a bone puncture or opening an anchor having an outer sleeve so that an outward extending flange disposed around the outer sleeve seats against cortical bone, the outer sleeve defining slots; advancing a fastener into an inner sleeve disposed inside the outer sleeve, so that fins move away from the longitudinal axis through respective slots; and engaging the fins with surrounding cancellous bone when expanded through respective slots in an extended mode. The anchor can be securely anchored in the bone such that the outward extending flange impairs further advancement into the bone or otherwise limits the depth of insertion, and the fins impair and/or prevent removal of the anchor from the bone.

In still a further embodiment, the method can include providing arms joined with respective fins and flexing the arms so that the fins move through the respective slots. The arms can be flexed via a fastener engaging the arms and pushing them outward as the fastener advances into a second opening of the inner sleeve. The arms can move toward an inner surface of the outer sleeve.

In yet a further embodiment, the method can include extending a suture from the anchor and above the opening defined in the bone, optionally before or after the fastener is installed and the fins are in the expanded mode; threading the suture through a soft connective tissue; pulling on the suture to advance the soft connective tissue toward the anchor; and securing the suture to hold the soft connective tissue in a predetermined orientation.

In still a further embodiment, the method can be used in connection with a rotator cuff repair surgical procedure. The bone can be a humerus bone, the soft connective tissue can be an end of a torn rotator cuff, and the end of the torn rotator cuff tendon can be engaged against the humerus bone as a result of the method using the anchor. In still yet a further embodiment, after identification of a need thereof, the method can be used on patients requiring revision of rotator cuff surgery, as well as biceps tendon repair of the humerus, Achilles tendon repair, triceps tend repair and/or patellar/quadriceps tendon repair.

The current embodiments provide a bone anchor and related method of use, which satisfactorily anchor an element, for example soft tissue, to bone. The anchors herein can improve the efficiency of the physician and can improve recovery time for the patient, reducing risk of anchor removal post-op. For patients with soft bone problems, such an anchor can minimize the number of anchors installed, reduce pain and reduce the incidence of repair failure. With such anchors, the sutures can hold the soft tissue well, thereby reducing the risk of having to repeat a particular surgery. The anchor and methods also can assist greatly with revision surgical procedures, such as revision rotator cuff surgery. Patients who are candidates for such revision procedures already have existing holes in their bone that have not healed yet, and thus can exhibit problems similar to patients with osteoporotic bone.

These and other objects, advantages, and features of the invention will be more fully understood and appreciated with reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a section view of the anchor of a current embodiment installed in bone with a fastener entering the anchor to expand the anchor to an expanded mode;

FIG. 2 is a section view of the anchor of FIG. 1 with fins extending from slots in a retracted mode;

FIG. 3 is a top perspective view of the anchor of FIG. 1;

FIG. 4 is a side view of the anchor of FIG. 1;

FIG. 5 is a top view of the anchor of FIG. 1;

FIG. 6 is a side view of the anchor of FIG. 1 being installed in bone;

FIG. 7 is a side view of the anchor of FIG. 1 installed in bone;

FIG. 8 is a perspective of a subject having a torn rotator cuff tendon;

FIG. 9 is a perspective view of a rotator cuff repair utilizing the anchor of FIG. 1 as a suture anchor in the subject;

FIG. 10 is a section view of another embodiment of an anchor;

FIG. 11 is an exploded side view of another embodiment of an anchor;

FIG. 12 is a section view of another embodiment of an anchor;

FIG. 13 is a top view of the anchor of FIG. 12;

FIG. 14 is a top perspective view of the anchor of FIG. 12; and

FIG. 15 is a side view of the anchor of FIG. 12.

### DESCRIPTION OF THE CURRENT EMBODIMENTS

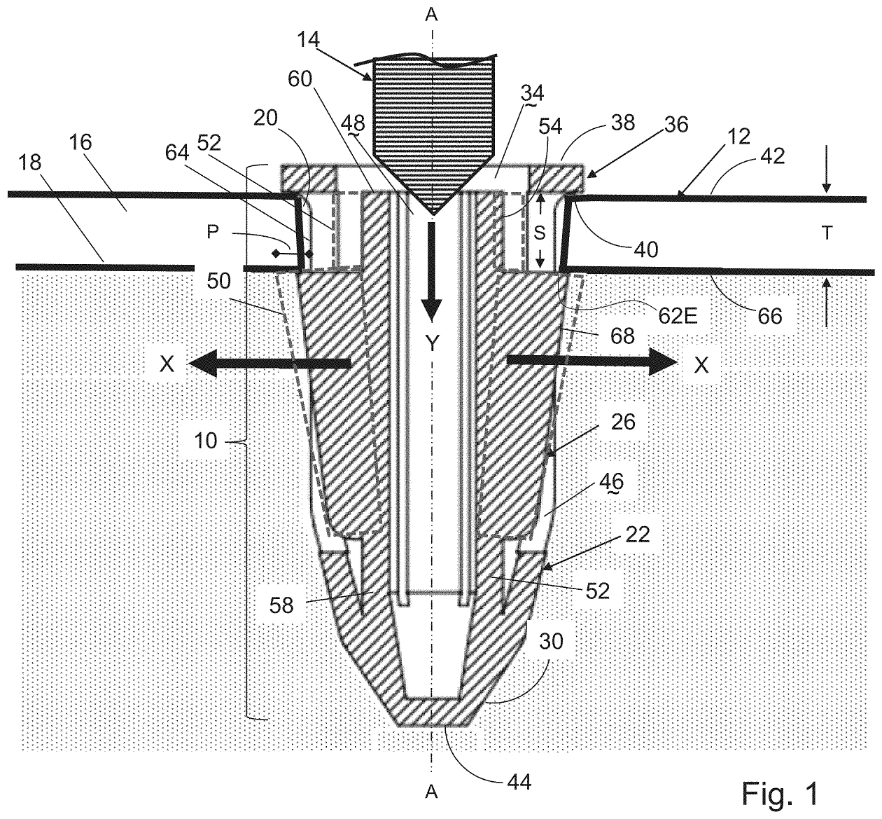

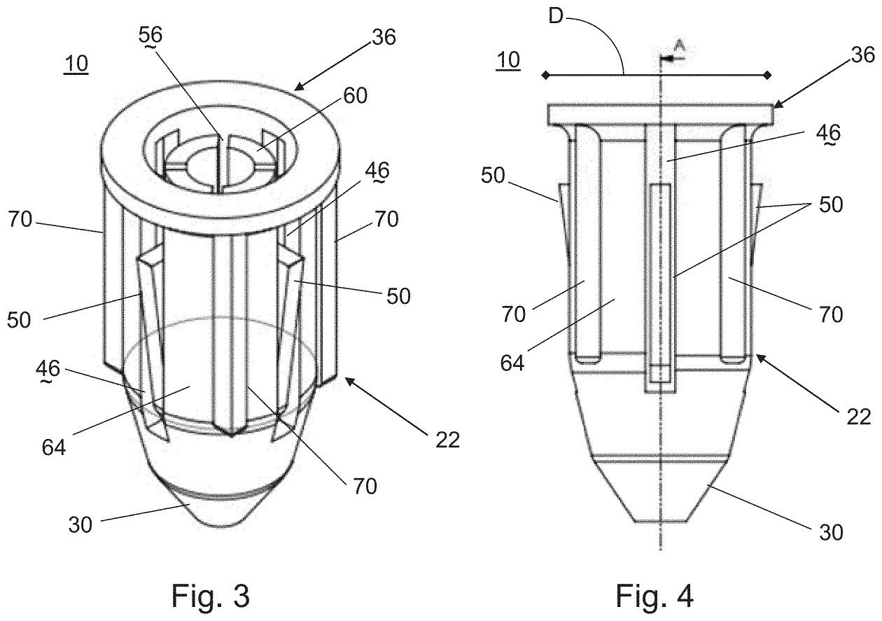

A current embodiment of the bone anchor is illustrated in FIGS. 1-5, and generally designated 10. In FIG. 1, the anchor 10 is shown installed in bone 12 with a fastener 14. More particularly, the bone 12 shown in FIG. 1 includes a hard outer layer of cortical bone 16 and internal tissue of cancellous bone 18, and an opening 20 in which the anchor 10 is at least partially installed. The opening 20 can extend through the layer of cortical bone 16 and into the cancellous bone 18, and can be a predrilled hole or puncture in the bone 12. In one example, such as during a rotator cuff repair, the bone 12 can be a humeral head or the humeral shaft. Together, the anchor 10 and fastener 14 can define an anchor assembly, with the anchor having a longitudinal axis A along which the fastener 14 can be inserted.

The anchor 10 can include one or more sleeves or sheaths. For example, it can include an outer sleeve 22 having an inner diameter defined by an inner surface 24 and an optional inner sleeve 26 disposed inside the outer sleeve 22, within the inner diameter or inner surface 24. The outer and inner sleeves 26 can be concentrically arranged on the longitudinal axis A. As shown herein, the inner sleeve 26 can be integrally formed with or otherwise joined to the outer sleeve 22 to effectively provide the anchor 10 as a one-piece article for a surgical procedure. As noted in other embodiments herein, the inner anchor can also be eliminated in some applications.

The outer sleeve 22 can include a distal end 28 having an insertion tip 30 and a proximal end 32 defining a first opening 34. An outward extending flange 36 is disposed around the first opening 34 and configured to seat against the cortical bone 16 at an entrance of the opening 20 and limit a depth of insertion of the outer sleeve 22 in the bone 12. The flange 36 can have an upper or proximal surface 38 and a lower or distal surface 40 that directly engages an outer surface 42 of the cortical bone 16. Optionally, the flange 36 can be spaced a preselected distance from the uppermost edges 62E of the fins 50. For example, the uppermost edges 62E of the fins 50 can each be spaced a preselected distance S from the distal surface 40. This distance S can be optionally greater than or equal to the thickness T of the cortical bone 16 adjacent to or at an entrance of an opening in the bone 12 so the fins 50 can expand, yet still provide a tight fit of the anchor 10 to the cortical bone 16. In the embodiment illustrated in FIG. 1, the distance S is shown as being substantially equal to or slightly greater than the thickness T of the cortical bone 16.

The insertion tip 30 can be tapered to facilitate insertion into the bone 12. As shown herein, the tapered insertion tip 30 angles inwardly, or toward from the longitudinal axis A, in the distal direction. The insertion tip 30 further includes a blunt distal-most end 44, but could have other profiles, including pointed or rounded. Optionally, the tip 30 can be pointed, and reinforced with a metal material, optionally in the form of a cone. This tip 30 can optionally facilitate puncturing of the bone 12 upon installation of the anchor 10.

The outer sleeve 22 can define a plurality of slots 46 radially disposed around the longitudinal axis A of the outer sleeve 22. The slots 46 can extend generally longitudinally, and may be substantially parallel to the longitudinal axis A. Further, the slots 46 can extend a substantial portion of the height or length of the outer sleeve 22, and can for example extend from above the insertion tip 30 to below the outward extending flange 36.

The inner sleeve 26 defines a second opening 48 aligned with the first opening 34 of the outer sleeve 22, and can include a plurality of fins 50 disposed below the outward extending flange 36 and radially around the longitudinal axis A. Each fin 50 can be aligned with a respective slot 46 of the outer sleeve 22, and can be radially movable away from the longitudinal axis A through the respective slot 46. As shown in FIG. 1, each fin 50 can be configured to project into and engage the cancellous bone 18 when expanded through the respective slot 46, as indicated by arrows X in FIG. 1. The anchor 10 can include an internal locking mechanism (not shown) for locking the fins 50 in the expanded position.

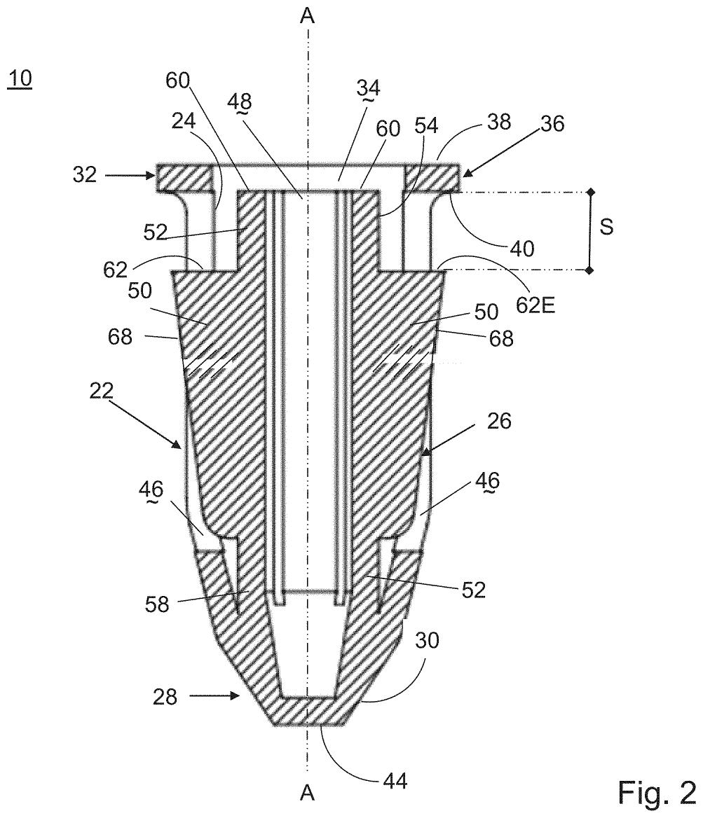

The inner sleeve 26 can include a plurality of arms 52 with which respective ones of the plurality of fins 50 are joined, formed, or otherwise carried therewith. The plurality of arms 52 can be configured to engage the inner surface 24 of the outer sleeve 22 when the plurality of fins 50 are in an expanded mode, one example of which is shown in FIG. 1 in phantom line. The arms 52 can be configured to flex inward toward the longitudinal axis A when the fins 50 engage cortical bone 16 around the opening 20 for the anchor 10 and/or as the anchor 10 is inserted into cancellous bone 18. In this state, or when the arms 52 are static and unflexed, the fins 50 can be in a retracted mode, one example of which is shown in FIG. 2. The arms 52 can be configured to flex or bend outward when the fastener 14 is installed. When the arms 52 bend outward, at least the fins 50 can engage and project into surrounding cancellous bone 18, as shown in phantom line in FIG. 1. In this state, the fins 50 can be in the extended mode. The direction of insertion of the fastener 14, indicated by arrow Y can be generally perpendicular to the direction of expansion indicated by arrows X.

In the illustrated embodiment, each of the plurality of slots 46 of the outer sleeve 22 is bordered by the inner surface 24, and each of the plurality of arms 52 includes an engagement surface 54 that is configured to engage the inner surface 24 when the plurality of fins 50 are in the expanded mode. The inner sleeve 26 optionally can be segmented to define the arms 52 arranged around the longitudinal axis A, and can include gaps 56 between the engagement surfaces 54 of adjacent arms 52. The gaps 56 extend to the proximal end of the inner sleeve 26, such that the inner sleeve 26 is discontinuous about the second opening 48.

Each of the plurality of arms 52 can include a distal end 58 and a proximal end 60, which collectively can define the second opening 48 of the inner sleeve 26. The distal ends 58 can define a base of the arms 52, which can be configured to flex so that each of the plurality of fins 50 can move outward through each respective slot 46. The distal ends 58 of the arms 52 can be joined with, such as being integrally formed with, the distal end 28 of the outer sleeve 22. As shown, the distal ends 58 of the arms 52 can join with an interior surface of the insertion tip 30, below the slots 46. The proximal ends 60 of the arms 52 are free or detached with respect to the outer sleeve 22 so that each of the fins 50 can move relative to their respective slot 46.

The plurality of fins 50 can be configured to engage the cortical bone 16 if extraction forces are exerted on the anchor 10. For example, each fin 50 can include an upper end 62, which can define a shoulder which extends substantially radially or normally from the engagement surface 54 of the arms 52. The upper ends 62 can protrude a preselected distance P from an outer surface 64 of the outer sleeve 22 in the extended mode, one example of which is shown in FIG. 1 in phantom line. This preselected distance P can be such that at least the upper end 62 extends beyond a radius or other dimension of the opening 20 in the bone 12. Thus, if the anchor 10 is pulled upward to dislodge it from the opening 20, the upper end 62 of one or more of the fins 50 can engage an inner surface 66 of the cortical bone 16, thereby impairing such removal.

The fins 50 can further include lateral surfaces 68 extending downwardly from the upper ends 62 meet the arms 52 near the base or distal end 58. The lateral surface 68 can be angled to facilitate insertion into bone 12, such as by being angled outwardly, or away from the longitudinal axis A, in a proximal direction from the tip 30 to the flange 36.

As shown in the embodiment illustrated, the second opening 48 can be recessed with respect to the first opening 34. As such, the upper ends 62 of the fins 50 are clear of the flange 36 for expansion during insertion of the fastener 14. The upper ends 62 of fins 50 are also shown as being recessed with respect to the proximal ends 60 of the arms 52 forming the inner sleeve 26, although in another configuration the fins 50 can extend to the proximal ends 60.

The optional outward extending flange 36 can be an annular flange and can extend around part or all of the outer sleeve 22. As shown in the illustrated embodiment, the annular flange 36 circumferentiates the outer sleeve 22, that is, the surrounds the outer sleeve 22. The diameter D of the outward extending flange 36 can define a maximum diameter of the outer sleeve 22, and of the anchor 10 overall, at least in the retracted mode of the fins 50, with the portion of the outer sleeve 22 depending below the outward extending flange 36 having a diameter no more than, and optionally less than, the diameter D of the outward extending flange 36. In some applications, the outward extending flange 36 can be eliminated from the anchor 10, in which case the outer sleeve 22 terminates at an upper edge of its body.

Optionally, the fins 50 can be selectively configured with regard to the outward extending flange 36. For example, when in the retracted mode, each fin 50 optionally does not extend beyond the outer edge or perimeter of the outward extending flange 36. In other cases, each fin 50 can extend the same distance or more from the longitudinal axis A as compared to a portion of the outward extending flange 36 above the particular fin 50. In other cases, different fins 50 can extend different distances, Some fins 50 can extend the same or a lesser distance than the outer edge of the outward extending flange 36, and others may extend a greater distance than the outward extending flange 36, away from the axis.

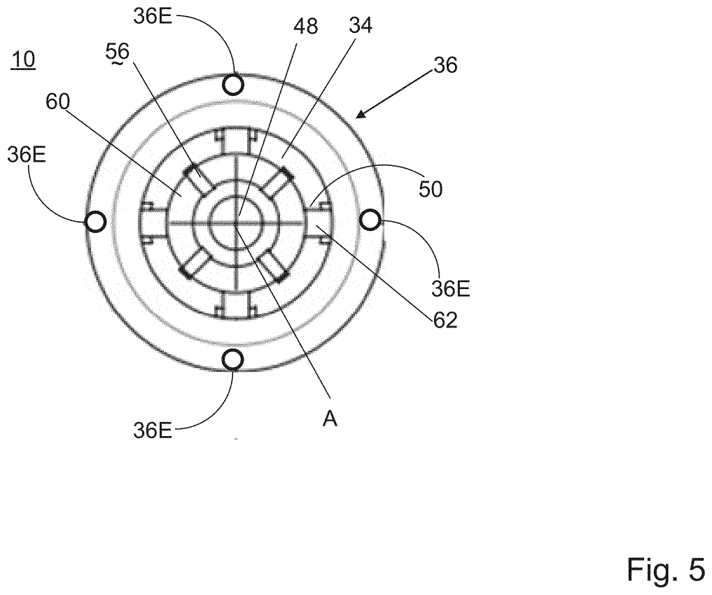

As shown in FIG. 5, the flange 36, or upper portion of the sleeve 26 when the flange is not present, can include indexing elements 36E. These indexing elements 36E can be indents, recesses, notches, protrusions, pimples or the like defined by or included in the flange 36 or sleeve 26, or can be in the form of paint, coatings, colored material or the like. These indexing elements 36E can be aligned with the fins 50 so that a user can determine the location of the fins 50 after the anchor 10 is installed in the bone 12, with the fins 50 no longer visible.

The fastener 14 is configured to be inserted into the first opening 34 and/or the second opening 48 and secured with the inner sleeve 26, thereby causing the plurality of fins 50 to move through the plurality of slots 46 so that the plurality of fins 50 enter into cancellous bone 18 a distance such that the plurality of fins 50 extend beyond a perimeter of the opening 20 in the bone 12. The fastener 14 has an outer diameter larger than an inner diameter of the inner sleeve 26 in the retracted mode such that insertion of the fastener 14 forces the arms 52 of the inner sleeve 26 apart, and projects the fins 50 through the slots 46 into the surrounding cancellous bone 18. In some cases herein, the term fastener can be used interchangeably with anchor.

Optionally, the outer surface 64 of the outer sleeve 22 can include a plurality of projections 70 extending outward therefrom under the outward extending flange 36. The projections 70 can be configured to engage the cancellous bone 18 and prevent rotation of the outer sleeve 22 relative thereto, that is, prevent rotation of the outer sleeve 22 about the longitudinal axis A relative to the opening 20 in the bone 12, such as during installation of the fastener 14 in the anchor 10. The projections 70 can be disposed between the fins 50 and/or between the slots 46 in the outer sleeve 22. The projections 70 can extend a preselected distance from the outer surface 64, which can be less than or equal to the distance P away from the outer surface 64 the fins 50 extend when the fins 50 are in the extended mode.

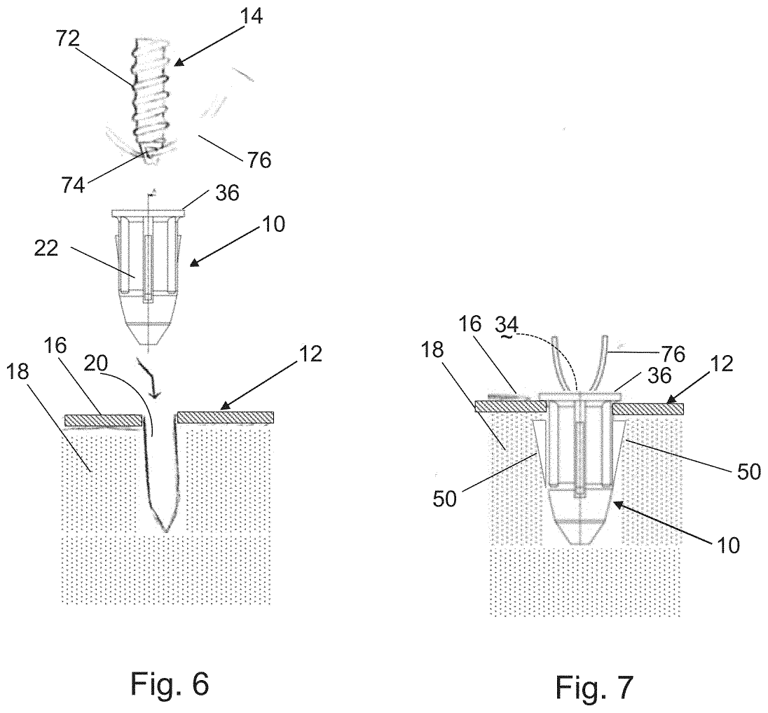

In one embodiment, as shown in FIG. 6, the fastener 14 can be sized to engage the inner sleeve 26 with a plurality of threads 72, and can define a hole 74 through which a suture 76 is threaded to secure the suture 76 to the fastener 14. Other embodiments of fasteners 14 for use with the anchor 10 are also possible. The inner sleeve 26 can be unthreaded to allow the anchor 10 to interact with fasteners having different types of threads. Thus, many existing fasteners can be used with the anchor 10. Some fasteners can be configured to be press fit into the sleeves 22, 26. Further, the fasteners can be slid, rotated, pounded, impacted or advanced in any manner into the sleeves 22, 26. Alternatively, the inner sleeve 26 can be provided with cooperating internal threads on its inner surface which mate with the external threads, ridges or projections 72 of the fastener 14.

The suture 76 can project from the first opening 34 after the fastener 14 is installed in the inner sleeve 26, as shown in FIG. 7. The suture 76 can be of a length configured to engage connective tissue to anchor the connective tissue to the bone 12 via the fastener 14 secured in the inner sleeve 26. In one example, such as during a rotator cuff repair, the connective tissue can be an end of a torn rotator cuff, with the suture 76 anchoring the an end of the torn rotator cuff to a humeral head via the fastener 14 secured in the inner sleeve 26.

The anchor 10, for example the sheath or the fastener, again, sometimes referred to as a fastener herein, can be made of nonabsorbable or bioresorbable material, including biocompatible metals, plastic, ceramics, and composites thereof. In one example, the anchor can be made of titanium, nitinol, stainless steel, polylactic acid (PLA, PLLA, or PLDLA), polyglycolic acid (PGA), polyether ether ketone (PEEK), or polyacetal. Other types of materials may be utilized in accordance with the invention, and the above are presented only as examples. The anchor 10 and its components optionally can include a coating or surface finish, such as an osteogenic coating or finish of bone growth factor. In other applications, the anchor and its components can be of a matrix design, with bone growth function compounds within the matrix.

A method of installing an anchor in bone will now be described with further reference to FIGS. 1 and 6-7. The sequence of steps discussed is for illustrative purposes only and is not meant to limit the method in any way as it is understood that the steps may proceed in a different logical order, additional or intervening steps may be included, described steps may occur simultaneously, sequentially, or may partially overlap, and/or described steps may be divided into multiple steps, without detracting from the invention. The method is described relative to the suture anchor 10 shown and/or described with reference FIGS. 1-5, although it is understood that the method may be applicable to other anchors.

The method can include inserting, into the opening 20 defined in the bone 12, the outer sleeve 22 so that the outward extending flange 36 seats against cortical bone 16. The outward extending flange 36 can limit a depth of insertion of the outer sleeve 22 in the bone 12. As described above, the opening 20 can be predrilled hole or puncture in the bone 12, and the method can optionally include appropriate steps for creating the opening 20 prior to the insertion of the anchor 10. An insertion tool (not shown) can be used to insert the anchor 10 into the bone 12.

The fastener 14 can be advanced into the inner sleeve 26 disposed inside the outer sleeve 22, so that each of the plurality of fins 50 moves away from the longitudinal axis A through a respective one of the plurality of slots 46. More specifically, the fastener 14 can be advanced into the first opening 34 of the outer sleeve 22 and into the second opening 48 of the inner sleeve 26. In an embodiment where the second opening 48 is recessed with respect to the first opening 34, as shown here, the fastener 14 enters the outer sleeve 22 before the inner sleeve 26, which can direct pressure distally on the anchor 10 prior to beginning expansion of the inner sleeve 26.

In an embodiment where the fastener 14 includes a plurality of threads 72, advancing the fastener 14 can include rotating the fastener 14. During the advancing, the fastener 14 can engage the inner sleeve 26 but not the outer sleeve 22, thereby causing the plurality of fins 50 to move through the plurality of slots 46 so that the plurality of fins 50 enter into the cancellous bone 18 a distance such that the plurality of fins 50 extend beyond a perimeter of the opening 50 defined in the bone 12.

As shown in FIGS. 1 and 7, each of the plurality of fins 50 can be engaged with cancellous bone 18 below the cortical bone 16, with each fin 50 expanded through a respective slot 46 in the extended mode. The anchor 10 can be securely anchored in the bone 12 such that the outward extending flange 36 impairs further advancement into the bone 12 and the plurality of fins 50 impairs removal of the anchor 10 from the bone 12. Upon engagement of one of the plurality of fins 50 with the cortical bone 18 opposite the outward extending flange 36, removal of the anchor 10 is impaired. Further, the anchor 10 also can engage the bone 12 at the opening 20 of the bone 12 to yield an interference fit there, which can further impair removal of the anchor 10 from the bone 12. Optionally, the cortical bone 16 can lie between the outward extending flange 36 and the upper ends 62 of the fins 50, and the engagement of the upper ends 62 against the inner surface 66 of the cortical bone 16 can impair removal of the anchor 10.

Optionally, where each of the plurality of fins 50 is joined with a corresponding flexible arm 52, the flexible arm 52 flexes by the advancement of the fastener 14 so that a respective joined fin 50 can move through a respective slot 46. As the fins 50 move radially away from the longitudinal axis A, the arms 52 can move toward the inner surface 24 of the outer sleeve 22. The engagement surfaces 54 can engage the inner surface 24 adjacent a respective slot 46 when the respective joined fin 50 is in the expanded mode shown in FIGS. 1 and 7.

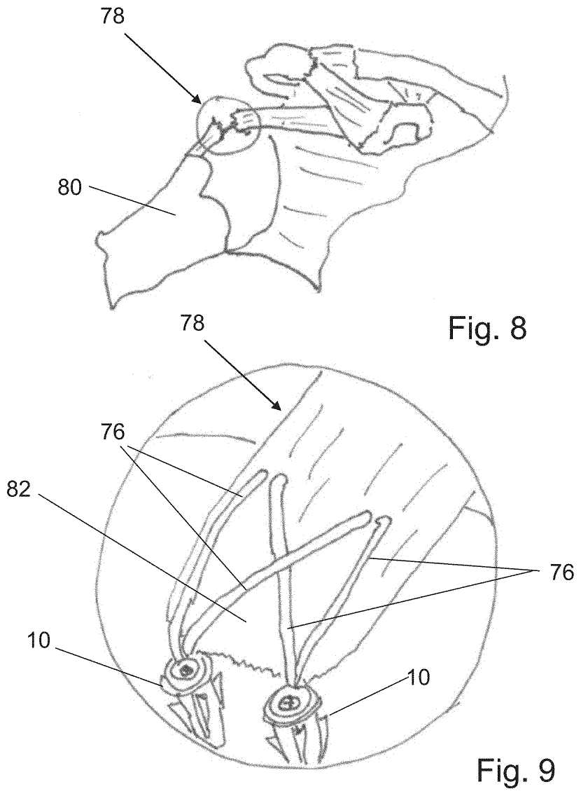

Further optionally, after the fastener 14 is installed and the fins 50 are in the expanded mode, a suture 76 can be extended from the anchor 14, above the opening 20 defined in the bone 12. In one example, where the fastener 14 includes defines a hole 74 through which the suture 76 is threaded to secure the suture 76 to the fastener, 14 the suture 76 is extended out of the first opening 34 and above the opening 20 in the bone 12.

Where a suture 76 is used, the method can include threading the suture 76 through a soft connective tissue, pulling on the suture 76 to advance the soft connective tissue toward the anchor 10, and tying or otherwise securing the suture to hold the soft connective tissue in a predetermined orientation. One example of this is shown in FIGS. 8-9 with respect to a rotator cuff repair. In FIG. 8, a perspective of a subject having a torn rotator cuff tendon is shown. During a rotator cuff repair, wherein the bone is a humerus bone 80, this can specifically entail threading the suture 76 though an end 82 of a torn rotator cuff 78, pulling on the suture 76 or advancing the fastener 14 into the anchor sleeves 22, 26 to advance the end 82 of the torn rotator cuff 78 toward the anchor 10, and optionally tying the suture 76 to hold the end 82 of the torn rotator cuff 78 against the humerus bone 80. FIG. 9 specifically shows a dual row repair in which two anchors 10 are used. The method can also be used on patients in need of revision rotator cuff surgery.

Optionally, the method can include utilizing the anchor 10 in a knotless, soft connective tissue repair procedure. In such a procedure, the surface 76 can be threaded through the soft connective tissue, which is pulled on advance the soft connective tissue toward the anchor 10, or a location where the anchor 10 is to be inserted into the bone. The anchor 10 can be installed in the bone opening if not already done. The suture 76 can be placed in the opening of the outer and/or inner sleeve 22, 26. The fastener can be advanced in those openings to thereby secure part of the suture 76 in the anchor 10, while the remaining suture limbs extend out of the bone to the soft connective tissue.

The suture anchor 10 was tested for failure mode and pullout strength using the following methodology. In particular, Arthrex SwiveLock® C anchors, available from Arthrex, Inc. (Naples, Fla.) were comparatively tested as installed alone, and alternatively as installed in the anchor 10 as shown in FIGS. 1-5. Ten 4.75 mm PLLA Arthrex SwiveLock® C anchors (“Arthrex Anchors”) provided in a kit with an accompanying tool and sutures were acquired to test the failure modes and pullout strength of the anchors 10 including the fasteners, that is the Arthrex anchors, and the sheath of the current embodiments. A representative model (ASTM D638 certified sheet) of cortical bone and a representative model (ASTM F-1839-08 certified foam block) of osteoporotic cancellous bone having a density of 10 lbs/ft³ were acquired from Sawbones (Vashon, Wash.), and were assembled together using a hot glue gun to create a bone model. Holes were drilled in the bone model to represent punctures from a surgeon. Five anchors 10 were 3D printed horizontally using FDM in acrylonitrile butadiene styrene (ABS) with a support structure, and were post-processed in a chemical bath to remove the support structure.

Five Arthrex anchors were screwed into the bone model without anchors 10 using the tool provided in the kit for the Arthrex anchors. Similarly, the five anchors 10 were then inserted into the drilled holes in the bone model. Once that was complete, five Arthrex anchors were screwed into those anchors 10, and in particular into the sheaths and in particular the inner sleeve thereof.

Next, each Arthrex anchor wall pulled out of the bone model by the sutures provided in the kit for the Arthrex anchors, using a MTS 858 Material Test System to provide a constant velocity pull of 1 cm per second. A force gauge was attached to the MTS machine to record the ultimate load tensile strength. The force gauge used was a Force Five Multi-Capacity force gauge, calibrated with a 1 kg weight, or 9.81 N.

The testing setup comprised a steel fixture screwed to a wooden panel which was fixed to the base of the MTS machine. The bone model was secured in the fixture in an orientation where the Arthrex anchors were substantially horizontal. When testing, the suture of the Arthrex anchor to be tested was fed through a slot in the fixture, around a channeled wheel, and tied to the force gauge. The wheel transformed the vertical pull of the MTS machine to a horizontal pull on the Arthrex anchor.

As a result of the testing, the anchors 10 of the current embodiments, including the Arthrex anchors and the sheath, exhibited superior and unexpected increased holding strength over the tested conventional Arthrex anchors by themselves. In particular, Table 1 displays the ultimate tensile strength and mode of failure for the pullout testing of the Arthrex anchors alone (#1-#5) and of the Arthrex anchors installed in the sheaths (#6-#10).

TABLE 1
Anchor Mode of Failure and Pullout Ultimate Tensile Strength.

| | | | Ultimate | |
|---|---|---|---|---|
| | | | Tensile | |
| Anchor No. | | Mode of Failure | Strength (N) | Notes |
| 1 | No sheath | Anchor pulled out | 5.10 | |
| 2 | No sheath | Anchor pulled out | 6.00 | |
| 3 | No sheath | Suture broke | 57.40 | Anchor stayed in bone model, was |
| | | inside anchor | | partially held behind cortical layer. |
| 4 | No sheath | Anchor pulled out | 2.00 | |
| 5 | No sheath | Anchor pulled out | 61.85 | Anchor was partially held behind |
| | | | | cortical layer. |
| 6 | With sheath | Suture broke | 37.20 | Sheath and anchor stayed in bone |
| | | inside anchor | | model |
| 7 | With sheath | Suture broke | 62.55 | Sheath and anchor stayed in bone |
| | | inside anchor | | model |
| 8 | With sheath | Suture broke | 43.30 | Sheath and anchor stayed in bone |
| | | inside anchor | | model |
| 9 | With sheath | Suture broke | 83.65 | Sheath and anchor stayed in bone |
| | | inside anchor | | model |
| 10 | With sheath | Suture broke | 112.10 | Sheath and anchor stayed in bone |
| | | inside anchor | | model |

For the Arthrex anchors alone, four of the five tests showed the failure due to the Arthrex anchor being pull out from the bone model, with one failure due to the suture breaking. This particular anchor (#3), was caught behind the cortical layer of the bone model as it was pulled out, which caused more resistance, and thus the higher value of ultimate tensile strength. This also occurred with #5. Each of the sheath and anchor combinations (#6-#10) had a failure mode of a broken suture, where the sheath and the anchor stayed in the bone model.

Generally, the ultimate tensile strength values of the sheath and anchor combinations (#6-#10) were an order of magnitude greater than that of the anchors alone (#1-#5). While #3 and #5 had higher values, this is likely due the anchors being partially held behind the cortical layer of the bone model during pull-out.

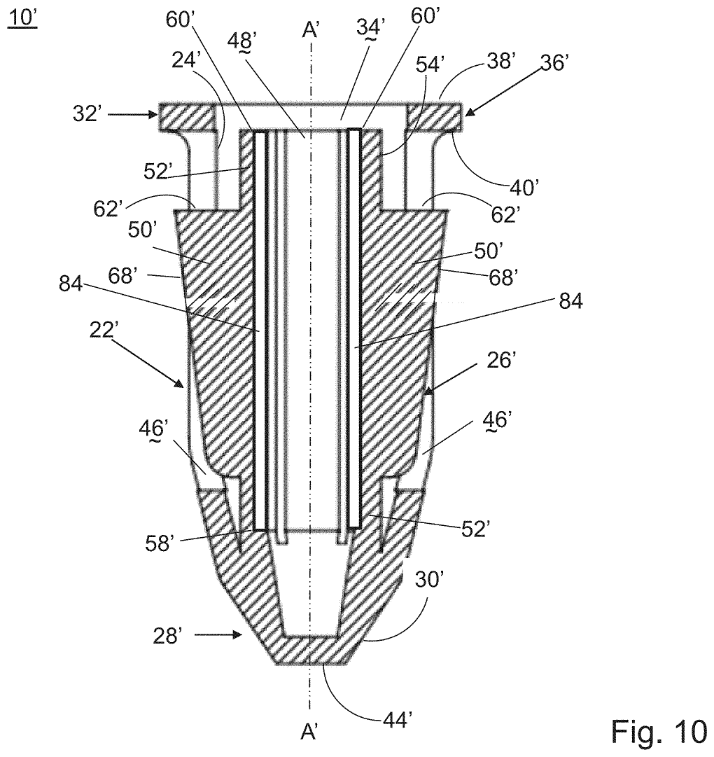

FIG. 10 shows another embodiment of a bone anchor 10′ in which like elements are identified using the same reference numerals bearing a prime (′) symbol. In FIG. 10, the anchor 10′ includes at least one channel 84 in the inner sleeve 26′ to accommodate sutures. This may be useful in combination with a fastener including a distal hole through which a suture is threaded to secure the suture to the fastener, as shown in FIG. 6. In the illustrated embodiment, opposing channels 84 are provided in the inner sleeve 26′ to accommodate two lengths of suture extending from a fastener.

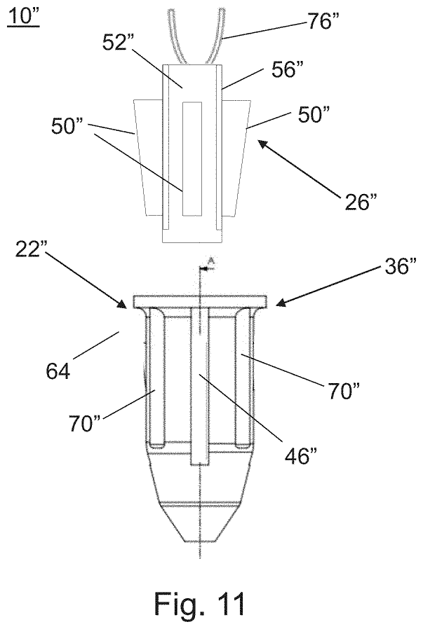

FIG. 11 shows another embodiment of a bone anchor 10″ in which like elements are identified using the same reference numerals bearing a double prime (″) symbol. In FIG. 11, the anchor 10″ includes separate outer and inner sleeves 22″, 26″. The outer sleeve 22″ is placed in the bone first, and includes projections 70″ or screw threads (not shown) to ensure the outer sleeve 22″ will remain well seated in the cortical bone. Next, the separate inner sleeve 26″, which carries sutures 76″, is inserted into the outer sleeve 22″ and the fins 50″ are expanded through the slots 46″ in the outer sleeve 22″ to make sure the inner sleeve 26″ remains well seated in the outer sleeve 22″ and does not rotate relative to the outer sleeve 22″. A fastener (not shown) can be used to expand the inner sleeve 26″ as described previously. Alternatively, the arms 52″ of the inner sleeve 26″ can be normally biased to an outward or expanded position such that the arms 52″ expand once the fins 50″ clear the outward extending flange 36″. The anchor 10″ can include an internal locking mechanism (not shown) for locking the fins 50″ in the expanded position.

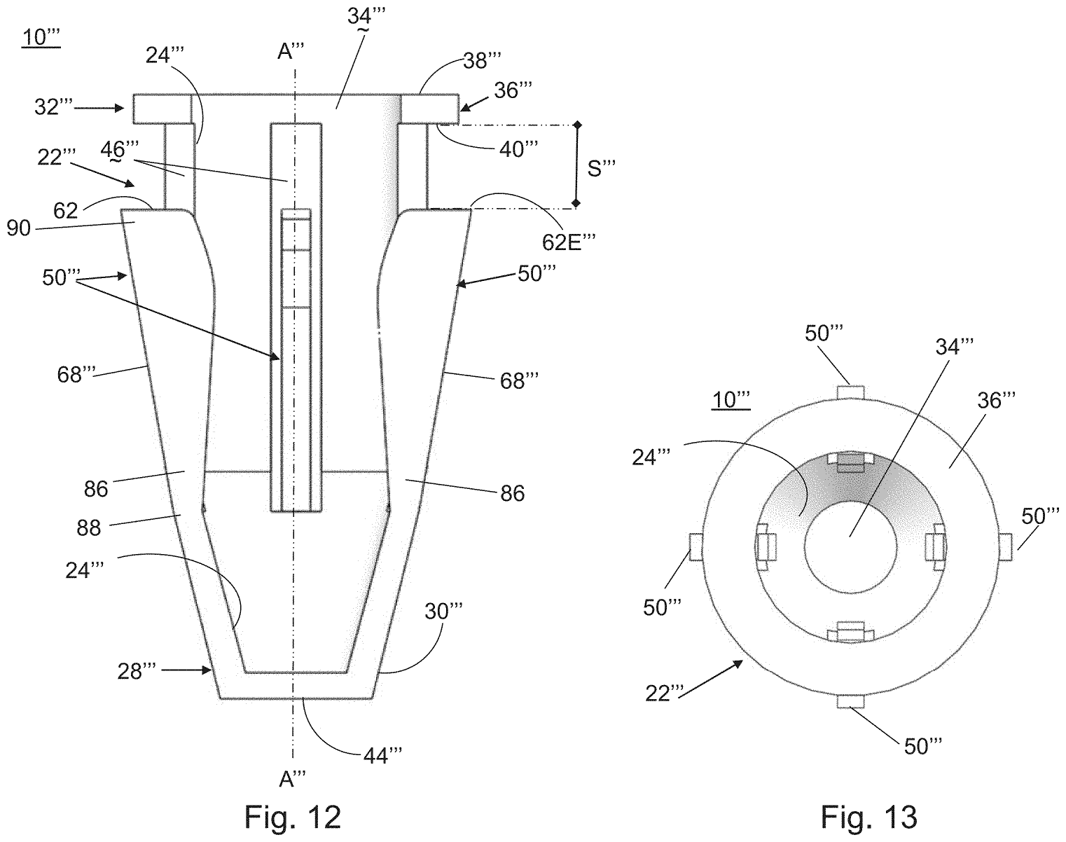

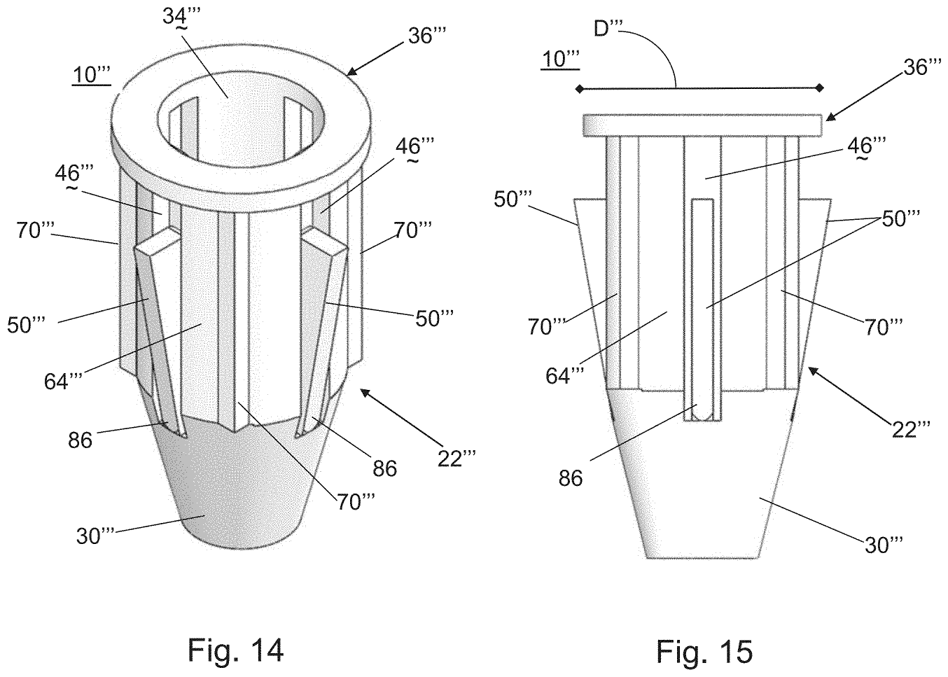

Another embodiment of a bone anchor 10′″ is illustrated in FIGS. 12-15, in which like elements are identified using the same reference numerals bearing a triple prime (′″) symbol. In this embodiment, the anchor 10′″ does not include an inner sleeve, and instead provides the fins 50′″ on the outer sleeve 22″, which is accordingly referred to in the present embodiment as simply sleeve 22′″. The fins 50′″ can be disposed below the outward extending flange 36′″ and radially around the longitudinal axis A′″. Each fin 50′″ can be aligned with a respective slot 46′″ of the sleeve 22″, and can be radially movable away from the longitudinal axis A′″, through the respective slot 46″, to project into and engage cancellous bone 18 when expanded. The anchor 10′″ can include an internal locking mechanism (not shown) for locking the fins 50′″ in the expanded position. Also, the flange 36′″ can include indexing elements (not shown) aligned with the fins 50′″ so that a user can determine the location of the fins 50′″ after the anchor 10′″ is installed in the bone, with the fins 50′″ no longer visible.

The sleeve 22′″ can include a plurality of arms 86 with which respective ones of the plurality of fins 50′″ are joined, formed, or otherwise carried therewith. The arms 86 can be configured to flex inward toward the longitudinal axis A′″ when the fins 50′″ engage cortical bone 16 around the opening 20 for the anchor 10′″ and/or as the anchor 10′″ is inserted into cancellous bone 18. In this state, or when the arms 86 are static and unflexed, the fins 50′″ can be in a retracted mode, one example of which is shown in FIG. 12. The arms 86 can be configured to flex or bend outward when the fastener 14 is installed. When the arms 86 bend outward, at least the fins 50′″ can engage and project into surrounding cancellous bone 18. In this state, the fins 50′″ can be in an extended mode. A fastener is configured to be inserted into the opening 34″, thereby causing the plurality of fins 50′″ to move through the plurality of slots 46′″.

Each of the plurality of arms 86 can include a distal end 88 and a proximal end 90. The distal ends 77 can define a base of the arms 86, which can be configured to flex so that each of the plurality of fins 50′″ can move outward through each respective slot 46′″. The distal ends 88 of the arms 52 can be joined with, such as being integrally formed with, the inner surface 24′″ of the sleeve 22″, near the distal end 28′″ or below the slots 46′″. The proximal ends 90 of the arms 52 are free or detached with respect to the proximal end 32′″ of the sleeve 22′″ so that each of the fins 50′″ can move relative to their respective slot 46′″.

For any of the embodiments of the anchor 10, 10′, 10″, 10′″ disclosed herein, a handheld insertion tool can be used to insert the anchor into the bone. The insertion tool can secure the anchor 10, 10′, 10″, 10′″ at a distal tip of the tool, and can have press button release or other actuator for release of the anchor 10, 10′, 10″, 10″. The anchor 10, 10′, 10″, 10′″ can be configured to interface with the insertion tool or release mechanism, such as having holes or other locator features provided thereon.

It is noted that the anchor 10, 10′, 10″, 10′″ shown and described herein could be used in procedures requiring bone anchors or bone screws without sutures secured thereto. In this case, a fastener can be provided, but need not secure a suture. Some non-limiting examples include screws for fixtures and devices, such as a bone support or a spinal support. Thus, the term “anchor” as used herein should not be construed as limiting the anchor to use with a suture, unless otherwise noted.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular. Any reference to claim elements as “at least one of X, Y and Z” is meant to include any one of X, Y or Z individually, and any combination of X, Y and Z, for example, X, Y, Z; X, Y; X, Z; and Y, Z.

1. A bone anchor comprising:
an outer sleeve including a distal end having an insertion tip and a proximal end defining a first opening, the outer sleeve including an inner diameter, the outer sleeve defining a plurality of slots radially disposed around a longitudinal axis of the outer sleeve, the outer sleeve configured to be placed adjacent cortical bone at an entrance of an opening in a bone; and
an inner sleeve disposed inside the outer sleeve, within the inner diameter, the inner sleeve defining a second opening aligned with the first opening, the inner sleeve including a plurality of fins disposed radially around the longitudinal axis, each fin being aligned with a respective slot, and radially movable away from the longitudinal axis through the slot, each fin being configured to project into and engage cancellous bone when each fin is expanded through the respective slot.

2. The bone anchor of claim 1 comprising:
an outward extending flange disposed around the first opening and configured to seat against cortical bone at the entrance of the opening in the bone and limit a depth of insertion of the outer sleeve in the bone,
wherein the plurality of fins are disposed a preselected distance below the outward extending flange, the distance configured to be less than or equal to a thickness of the cortical bone adjacent the entrance of the opening in the bone.

3. The bone anchor of claim 1 comprising:
a fastener sized to engage the inner sleeve with a plurality of threads, the fastener defining a hole through which a suture is threaded to secure the suture to the fastener, the fastener configured to be inserted into the first opening and the second opening, thereby causing the plurality of fins to move through the plurality of slots so that the plurality of fins enter into cancellous bone a distance such that the plurality of fins extend beyond a perimeter of an opening in the bone.

4. The bone anchor of claim 3,
wherein the plurality of fins are configured to engage cortical bone if extraction forces are exerted on the suture anchor.

5. The bone anchor of claim 4,
wherein the suture projects from the first opening after the fastener is installed in the inner sleeve,
wherein the suture is of a length configured to engage connective tissue to anchor the connective tissue to the bone via the fastener secured in the inner sleeve.

6. The bone anchor of claim 1,
wherein the inner sleeve includes a plurality of arms with which respective ones of the plurality of fins are joined,
wherein the plurality of arms are configured to engage an inner surface of the outer sleeve when the plurality of fins are in an expanded mode.

7. The bone anchor of claim 6,
wherein each of the plurality of slots is bordered by an inner surface of the outer sleeve,
wherein each of the plurality of arms includes an engagement surface that is configured to engage the inner surface when the plurality of fins are in an expanded mode,
wherein each of the plurality of arms includes a base that is configured to flex so that each of the plurality of fins can move outward through each respective slot.

8. The bone anchor of claim 1,
wherein the inner sleeve is segmented to include a plurality of arms arranged around the longitudinal axis,
wherein each arm includes a respective one of the plurality of fins,
wherein the outer sleeve includes an outer surface,
wherein the outer surface includes a plurality of projections extending outward therefrom under the outward extending flange,
wherein the projections are configured to engage cancellous bone and prevent rotation of the outer sleeve relative thereto.

9. A bone anchor comprising:
a sleeve including a distal end having an insertion tip and a proximal end defining a first opening configured to receive a fastener, the sleeve defining at least one slot disposed outwardly from a longitudinal axis of the sleeve, the sleeve configured to be placed adjacent a cortical bone at an entrance of an opening in a bone, the sleeve including at least one fin,
wherein the at least one fin is aligned with the at least one slot and movable away from the longitudinal axis, upon engagement with the fastener, within the slot,
wherein the at least one fin is configurable in an extended mode in which the at least one fin projects into and engage cancellous bone when the fin is extended through the at least one slot.

10. A method of installing an anchor in bone comprising:
inserting into an opening defined in a bone an outer sleeve defining a plurality of slots disposed around a longitudinal axis of the outer sleeve;
advancing a fastener into the first opening and a second opening of an inner sleeve disposed inside the outer sleeve, so that each of a plurality of fins, disposed below the outward extending flange and radially around the longitudinal axis, moves away from the longitudinal axis through a respective one of the plurality of slots,
engaging each of the plurality of fins with cancellous bone below the cortical bone when each fin is expanded through a respective slot in an extended mode,
wherein the anchor is securely anchored in the bone such that the plurality of fins impairs removal of the suture anchor from the bone upon engagement of one of the plurality of fins with at least one of cancellous bone and the cortical bone.

11. The method of claim 10,
wherein an outward extending flange disposed around a first opening of the outer sleeve seats against cortical bone,
wherein the outward extending flange impairs farther advancement of the outer sleeve into the bone.

12. The method of claim 10,
wherein the fastener includes a plurality of threads,
wherein the advancing includes rotating the fastener,
wherein the fastener defines a hole through which a suture is threaded to secure the suture to the fastener,
wherein the fastener engages the inner sleeve during the advancing but not the outer sleeve, thereby causing the plurality of fins to move through the plurality of slots so that the plurality of fins enter into the cancellous bone a distance such that the plurality of fins extend beyond a perimeter of the opening defined in the bone.

13. The method of claim 12,
wherein the suture extends out of the first opening and above the opening defined in the bone after the fins are in the expanded mode.

14. The method of claim 13, comprising:
threading the suture through a soft connective tissue; and
pulling on the suture to advance the soft connective tissue toward the anchor, and thereby hold the soft connective tissue in a predetermined orientation.

15. The method of claim 14,
wherein the bone is a humerus bone,
wherein the soft connective tissue is an end of a torn rotator cuff,
wherein the end of the torn rotator cuff is engaged against the humerus bone after the advancing.

16. The method of claim 10, comprising:
wherein each of the plurality of fins is joined with a corresponding flexible arm,
wherein the flexible arm flexes so that a respective joined fin can move radially away from the longitudinal axis and through a respective slot.

17. The method of claim 15,
wherein the arm includes an engagement surface that engages an inner surface of the outer sleeve adjacent a respective slot when the respective joined fin is in an expanded mode.

18. A method of installing an anchor in bone comprising:
inserting into an opening defined in a bone an outer sleeve so that an outward extending flange disposed around a first opening of the outer sleeve seats against cortical bone, the outer sleeve defining a plurality of slots;
advancing a fastener into an inner sleeve disposed inside the outer sleeve, so that each of a plurality of fins, disposed below the outward extending flange moves away from the longitudinal axis through a respective one of the plurality of slots,
engaging each of the plurality of fins with cancellous bone when each fin is expanded through a respective slot in an extended mode,
wherein the anchor is securely anchored in the bone such that the outward extending flange impairs further advancement into the bone and the plurality of fins impairs removal of the suture from the bone.

19. The method of claim 18 comprising:
providing a plurality of arms joined with respective ones of the plurality of fins;
flexing the plurality of arms so that the plurality of fins move through the respective slots;
wherein the arms move toward an inner surface of the outer sleeve.

20. The method of claim 18, comprising:
extending a suture from the anchor and above the opening defined in the bone after the fastener is installed and the fins are in the expanded mode;
threading the suture through a soft connective tissue;
pulling on the suture to advance the soft connective tissue toward the anchor; and
securing the suture to hold the soft connective tissue in a predetermined orientation.
